(19) United States
(12) Patent Application Publication
(10) Pub. No.: US 2023/0404956 A1
(43) Pub. Date: Dec. 21, 2023

(54) THERAPEUTIC WINDOW FOR CITRULLINE THERAPY

(71) Applicants: Asklepion Pharmaceuticals LLC, Baltimore, MD (US); Vanderbilt University, Nashville, TN (US)

(72) Inventors: Gurdyal Kalsi, Potomac, MD (US); Marshall Summar, Washington, DC (US); Frederick E. Barr, Little Rock, AR (US); Omar Ayyub, Washington, DC (US); Gary R. Pasternack, Baltimore, MD (US)

(21) Appl. No.: 17/807,975

(22) Filed: Jun. 21, 2022

(51) Int. Cl. A61K 31/198; A61K 35/14; A61P 41/00

(52) U.S. Cl. CPC A61K 31/198; A61K 35/14; A61P 41/00

(57) ABSTRACT

This invention is directed to citrulline therapy, where citrulline is administered to achieve a newly recognized therapeutic window. The results of a completed clinical trial has elucidated a therapeutic window for L-citrulline. Detailed analysis of clinical outcome as a function of the dose actually received reveals that patients falling within the dosing window showed significant improved effect compared to patients receiving a dose either below or above the dosing window. Patients benefiting from this invention are patients in need of supplemental citrulline to ensure that adequate nitric oxide levels are produced by endothelial tissues.

This invention is directed to citrulline therapy, where citrulline is administered to achieve a newly recognized therapeutic window. Patients benefiting from this invention are patients in need of supplemental citrulline to ensure that adequate nitric oxide levels are produced by endothelial tissues.

### BACKGROUND

Cardiopulmonary bypass (CPB) induced injury results from a principally humoral systemic inflammatory response induced by the bypass process. Seghaye (2003) Cardiol Young 13(3): 228-239; Day & Taylor (2005) Int J Surg 3(2): 129-140; Jaggers & Lawson (2006) Ann Thorac Surg 81(6): S2360-2366; Kozik & Tweddell (2006) Ann Thorac Surg 81(6): S2347-2354; Warren et al. (2009) J Cardiothorac Vasc Anesth 23(2): 223-231; Warren et al. (2009) J Cardiothorac Vasc Anesth 23(3): 384-393. The damage sustained by the lung and other tissues results in the serious clinical condition described below.

Increased postoperative pulmonary vascular tone (PVT) is an increase in the contraction of smooth muscle in vessel walls. Increased PVT is a common complication after repair of a variety of congenital heart defects. Steinhorn, et al. Artificial Organs (1999) 23: 970-974; Schulze-Neick, et al. J Thorac Cardiovasc Surg (2001) 121: 1033-1039. The pathophysiology of increased postoperative PVT is believed to be involved in pulmonary vascular endothelial cell dysfunction. Steinhorn, et al. Artificial Organs (1999) 23: 970-974. Limited studies have been performed on the effects that cardiopulmonary bypass (CPB) has on pulmonary endothelial function. In a study of 10 infants undergoing CPB for repair of congenital defects, supplementation of the NO precursor, arginine, ameliorated pulmonary endothelial dysfunction. Schulze-Neick, et al. Circulation (1999) 100: 749-755. In animal studies, endothelial cell production of NO is diminished after cardiopulmonary bypass but still a major controlling factor with respect to pulmonary vasomotor tone. Kirshbom, et al. J thorac Cardiovasc Surg (1996) 111: 1248-1256.

Endogenous nitric oxide (NO) plays a role in the regulation of pulmonary vascular tone. Nelin, et al. Pediatr Res (1994) 35: 20-24; Lipsitz, et al. J Pediatr Surg (1996) 31: 137-140. Nitric oxide is synthesized by different isoforms of the enzyme nitric oxide synthase (NOS). Endothelial NOS (eNOS) is a constitutive enzyme responsible for the calcium-calmodulin dependent production of baseline levels of NO. Inducible NOS (iNOS) catalyzes the calcium-independent production of large amounts of NO in response to certain cytokines and inflammatory stimuli. A third form of NOS is neuronal NOS (nNOS) serves as a neurotransmitter in both the central and peripheral nervous systems. Endothelial cells generate endogenous NO from arginine as the proximal substrate, but use citrulline as the ultimate precursor for NO production. Palmer, et al. Biochem Biophys Res Commun (1988) 153: 1251-6; Moncada, et al. N Engl J Med (1993) 329: 2002-12.

The hepatic urea cycle plays a role in the production of two precursors of nitric oxide: arginine and citrulline. Pearson, et al. New England Journal of Medicine (2001) 344: 1832-1838. The first two steps of the hepatic urea cycle, carried out by carbamyl phosphate synthetase I (CPSI) and ornithine transcarbamylase (OTC), produce citrulline. These two enzymes are located in mitochondria of the liver and gut with the remainder of the pathway distributed throughout the body, including the pulmonary vascular endothelium. Summar J Inher Metab Dis (1988) 21(S1): 30-39. Citrulline, an amino acid, is the first intermediate of the urea cycle. After citrulline is transported intracellularly via a selective membrane transporter, it is rapidly converted to arginine by the enzymes argininosuccinate synthetase (AS) and argininosuccinate lyase (AL).

Pulmonary vascular tone can be an important perioperative issue, even for patients not at risk for pulmonary artery hypertension. Protocols for maintaining postoperative pulmonary vascular tone (i.e., reducing or eliminating any increase in PVT) may be helpful in decreasing the need for prolonged mechanical ventilation, ICU stay, and hospitalization. Thus, there exists a need in the art for improvement in the system for maintaining pulmonary vascular tone in a patient during surgery and postoperatively.

### SUMMARY OF THE INVENTION

This invention is directed to treatment methods encompassing a therapeutic window for citrulline therapy. In particular, the invention provides a method of maintaining nitric oxide balance in a mammal in need thereof, the method comprising administering citrulline to the mammal such that the plasma citrulline concentration is maintained between 80-180 μM. According to this invention, the citrulline may be administered via intravenous bolus or infusion, orally, or via ultrafiltration in hemoconcentration replacement fluid. In various embodiments, the mammal in need of nitric oxide balance suffers from hypocitrullinemia, is at risk for postoperative pulmonary hypertension, is at risk for acute right-sided heart failure, tricuspid regurgitation, systemic hypotension, myocardial ischemia, and/or increased airway resistance. In other embodiments, the mammal in need suffers from sickle cell disease, especially sickle cell crisis, subarachnoid hemorrhage, bronchopulmonary dysplasia, pulmonary hypertension, (including primary and secondary), hyperammonemia, asthma, and/or other urea cycle related conditions. Other representative disorders and conditions that may be expected to benefit from administration of citrulline within the therapeutic window of this invention include but are not limited to hepatitis (including hepatitis A, B and C), cirrhosis, sclerosis, asthma, especially acute asthma attack sometimes termed status asthmaticus, Acute Respiratory Distress Syndrome (ARDS), bone marrow transplant toxicity in a subject undergoing bone marrow transplant, chemotherapy or other pharmaceutical therapy, sepsis, ethnic specific endothelial dysfunction, erectile dysfunction, priapism, necrotizing enterocolitis (NEC), increased oxidative stress, sepsis, hypoxia, hepatotoxin exposure, and combinations thereof. In various embodiments of this invention, the plasma citruline concentration is maintained between 80-160 μM. Preferably, citrulline administration is aimed at achieving a target plasma concentration of approximately 120 μM.

In particular embodiments, this invention provides a method for maintaining the coupling of endothelial nitric oxide synthase (eNOS) to reduce the incidence or severity of cardiopulmonary bypass-induced pulmonary injury due to free radical formation in a patient during cardiopulmonary bypass comprising administering an amount of citrulline to the patient, effective such that the plasma citrulline concentration of the patient is maintained between 80-180 μM. The method of this invention may also include a step of administering citrulline to the patient during the surgery. The method of this invention includes a step where citrulline administered during the surgery, preferably citrulline is added to the cardiopulmonary bypass filtration and/or the hemoconcentration replacement fluid. Preferably, citrulline is administered to the patient in an amount to achieve about 80-180 μmol/L in plasma, more preferably about 80-160 μmol/L. In preferred embodiments, plasma citrulline target level is about 120 μmol/L.

### BRIEF DESCRIPTION OF THE FIGURES

FIG. 1 shows results for the composite endpoint for the complete Modified Full Analysis Set. The Modified Full Analysis Set consists of all enrolled patients who completed the protocol.

FIG. 2 shows composite endpoint vs % dose 2 administered for the complete modified full Analysis set. Note that patients receiving the full calculated dose appear to do less well than patients receiving lower dose levels.

FIG. 3 shows the composite endpoint exhibits highly significant differences between placebo and control in patients receiving <75% of the calculated intraoperative Dose 2 of citrulline. 13 outlier data points above 7500 minutes are not shown, but are included in the analysis.

FIG. 4 shows differences in the duration of mechanical ventilation between placebo and control in patients receiving <75% of the calculated intraoperative Dose 2 of citrulline for the modified full analysis set. Seven outlier data points above 7500 minutes are not shown, but are included in the analysis.

FIG. 5 shows difference in duration of inotrope therapy between placebo and control in patients receiving <75% of the calculated intraoperative Dose 2 of citrulline for the modified full analysis set. Eight outlier data points above 7500 minutes are not shown, but are included in the analysis.

FIG. 6 shows difference in duration of PICU/NICU stay between placebo and control in patients receiving <75% of the calculated intraoperative Dose 2 of citrulline for the modified full analysis set. One outlier datapoint is excluded from the graph but not the analysis. Data was missing for two patients, who were excluded from the analysis.

FIG. 7 shows difference in duration of hospital stay between placebo and control in patients receiving <75% of the calculated intraoperative Dose 2 of citrulline for the modified full analysis set. One outlier datapoint is excluded from the graph but not from the analysis.

FIGS. 8A-8C show patients receiving >95% of the targeted dose had worse results when treated with citrulline than placebo patients for (A) the composite variable, (B) duration of mechanical ventilation, and (C) duration of inotrope therapy. These results were highly statistically significant. Outliers exceeding 7500 minutes were excluded from the graphs for presentation purposes, but not from the analysis. Points so excluded were: 19, composite time; 12, vent time; and 11, inotrope time.

### DETAILED DESCRIPTION OF THE EMBODIMENTS

The results of a completed Phase 3 trial using data on all patients at all sites has elucidated a therapeutic window for L-citrulline. Detailed analysis of clinical outcome as a function of the intraoperative dose actually received has revealed a window that fully explains the results. Patients falling within the dosing window showed significant effect not only for SPA-agreed¹ primary endpoints—duration of mechanical ventilation and that of inotrope therapy—but for direct measurement of pediatric intensive care unit (PICU) time as well. The data and analyses confirm not only that L-citrulline has a therapeutic effect on the post-operative status and prognosis of pediatric patients exposed to cardiopulmonary bypass in congenital heart defect surgery, but further substantiates the proposed effective dose range. ¹ Under the FDA's Special Protocol Assessment (SPA) procedure, an agreement via the SPA procedure is an agreement with the FDA that a Phase 3 clinical trial's design (e.g., endpoints, sample size, control group and statistical analyses) is acceptable to support a regulatory submission seeking new drug approval.

Phase 2 Study

A Phase 2 study was carried out showing the effect of administering citrulline to pediatric patients undergoing surgical procedures involving cardiopulmonary bypass. The results of the phase 2 trial are described in U.S. Pat. No. 10,265,286, which is incorporated herein by reference in its entirety. The phase 2 double-blind placebo-controlled trial involved 22-patients (11 Tx, 11PL), and showed near-significance in the primary parameters (duration of MV & InoTx)². In the phase 2 study, the Intraoperative dose (Dose 2) was 100 μmol. ² Duration of mechanical ventilation and that of inotrope therapy.

Phase 3 Study

A Phase 3 study was carried out as a double-blind, placebo-controlled trial to confirm the effect. The Phase 3 trial was performed in the US, EU, and Israel, using a modified intent-to-treat population (or modified full analysis set) of 190 patients, of whom 138 were in the US and 52 were ex-US. The demographics of the US and ex-US populations were comparable.

Unexpectedly, especially after the preliminary evidence of effect in the earlier Phase 2 Trial, initial analysis per the methods in the Statistical Analysis Plan (SAP, approved under the SPA agreement) failed to reveal statistically significant differences in primary endpoints for the treated and placebo populations.

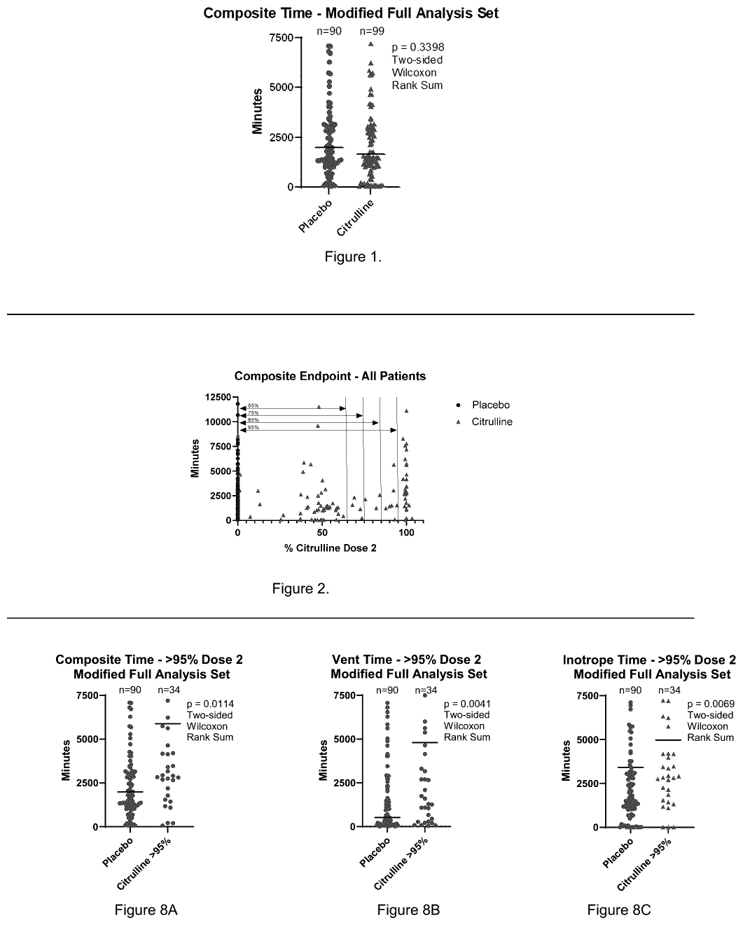

The starting point for the analysis was consideration of the complete modified full analysis set, consisting of all enrolled patients who completed the protocol. The results of the composite endpoint (the greater of length of time on mechanical ventilation or length of time on inotrope therapy) agreed to with FDA under SPA showed no difference, as seen in FIG. 1, which shows Results for the composite endpoint for the complete Modified Full Analysis Set. The Modified Full Analysis Set consists of all enrolled patients who completed the protocol.

Re-Analysis of Phase 3 Study Data

In the study protocol and accompanying pharmacy manual for both studies, citrulline dosing occurs in four distinct phases. Dose 1 is a loading dose added to the cardiopulmonary bypass fluids on a mg/kg basis. Dose 3 is a bolus administered following surgery on a mg/kg basis, and Dose 4 is an IV drip at 9 mg/kg/hour. No variation in administration of these doses was introduced during the course of the phase 3 study.

In contrast, Dose 2, the intraoperative dosing designed to compensate for ultrafiltration, was subject to some variation. Ultrafiltration during cardiac surgery is not standardized, and therefore, it was not possible to completely standardize this dose. The perfusionist estimates the volume of ultrafiltrate removed, as well as the volume of cardioplegia solution. The perfusionist then calculates according to the study pharmacy manual instructions the amount of citrulline to be administered to compensate for the ultrafiltered citrulline in order to maintain a specified therapeutic level.

Although the therapeutic target level was set at 100 micromolar during the phase 2 study, because of the potential for variation in Dose 2 (which is given intraoperatively to compensate for the routine ongoing ultrafiltration of circulating bypass fluids to reduce the circulating concentrations of inflammatory mediators) and the concern that some patients might inadvertently receive sub-therapeutic intraoperative levels, the target level for Dose 2 in the phase 3 study was increased to 200 micromolar. This was considered to be safe since no adverse effects of citrulline had been reported, even at substantially higher levels in medical use. In order to examine whether variation in Dose 2 could explain the results obtained, the actual dose received, shown as the percent of the targeted dose, was plotted against the composite variable results. Since placebos received no citrulline, they were plotted at the 0% level.

For the Phase 3 trial, a target intraoperative dose of 200 μmol (Dose 2) was chosen to preclude an inadvertent drop below an efficacious level in any patient. The potential for variation in intraoperative dosing arises from the lack of standardization of cardiopulmonary bypass ultrafiltration techniques. In the Phase 2 study, Dose 2 variation did not result in any observed problems or loss of efficacy. Nonetheless, expansion of the study to a greater number of sites raised the concern that some sites might inadvertently underdose Dose 2. Given the known innocuous nature of L-citrulline, an endogenous amino acid that is given medicinally in doses an order of magnitude higher than this to urea cycle disorder patients, as well as the well-tolerated high endogenous concentrations of citrulline in hypercitrullinemia patients, there was no concern for erring on the high side of the presumed effective dose.

To investigate the data in more detail, patients were stratified according to the dose level actually administered intraoperatively. The values obtained for duration of mechanical ventilation (MV), length of inotrope therapy (InoTx) and the composite primary (created for the SAP, based on the longer of the MV or InoTx) in any patient were segmented into groups based on the percent of the 200 μmol target dose actually received by the patient, by comparing patients who had received <65%, <75%, <85%, <95%, and >95% of the calculated target dose. Statistically significant reduction of all variables was seen for all doses under 95% of the target. Analysis was performed via two-sided Wilcoxon Rank Sum. Patients receiving respectively <95%, <85%, <75%, and <65% were analyzed in comparison to placebos as well as patients receiving >95% (see FIG. 2 and Table 1 below).

TABLE 1
Median values for all primary and secondary endpoints at all dose stratifications.
Statistical analyses performed utilizing two-sided Wilcoxon Rank Sum.

| Endpoint | Placebo | All Patients | <95% Dose | <85% Dose | <75% Dose | <65% Dose | >95% Dose |
|---|---|---|---|---|---|---|---|
| Composite | 2204.0 | 1640.0 | 1357.0 | 1283.5 | 1267.0 | 1267.0 | 3447.0 |
| (minutes) | | (p = 0.3398) | (p = 0.0033) | (p = 0.0029) | (p = 0.0025) | (p = 0.0040) | (p = 0.0114) |
| Vent | 574.5 | 672.0 | 237.0 | 176.0 | 170.0 | 139.0 | 2385.0 |
| (minutes) | | (p = 0.9205) | (p = 0.0459) | (p = 0.0163) | (p = 0.0129) | (p = 0.0097) | (p = 0.0041) |
| Inotrope | 1717.0 | 1514.0 | 1300.0 | 1263.5 | 1260.0 | 1260.0 | 3142.5 |
| (minutes) | | (p = 0.7131) | (p = 0.0242) | (p = 0.0176) | (p = 0.0158) | (p = 0.0211) | (p = 0.0069) |
| PICU/NICU | 3 | 3 | 2 | 2 | 2 | 2 | 4 |
| (days) | | (p = 0.8484) | (p = 0.1232) | (p = 0.0332) | (p = 0.0295) | (p = 0.0236) | (p = 0.0034) |
| Hospitalization | 5 | 5 | 5 | 5 | 5 | 4 | 7 |
| (days) | | (p = 0.3681) | (p = 0.3169) | (p = 0.1369) | (p = 0.1905) | (p = 0.0953) | (p = 0.0006) |

FIG. 2 shows composite endpoint vs % dose 2 administered for the complete modified full Analysis set. Since placebo controls received no citrulline, the value for Dose 2 was set to zero for these patients. Note that patients receiving the full calculated dose appear to do less well than patients receiving lower dose levels.

The resultant analyses demonstrated that administration of Dose 2 at levels <95% showed highly significant reductions in the composite variable, the duration of mechanical ventilation, and the duration of inotrope therapy. Moreover, the data further showed a statistically significant reduction of one day in the length of stay in the PICU or NICU following surgery.

Identification of Therapeutic Window

Unexpectedly, the analyses of the phase 3 trial data showed a statistically significant negative effect at doses higher than 95% of target (i.e., >190 μmol), as shown in FIGS. 8A-8C. FIGS. 8A-8C show that patients receiving >95% of the targeted dose had worse results when treated with citrulline than placebo patients for (A) the composite variable, (B) duration of mechanical ventilation, and (C) duration of inotrope therapy. These results were highly statistically significant. Outliers exceeding 7500 minutes were excluded from the graphs for presentation purposes, but not from the analysis. Points so excluded were: 14, composite time; 8, vent time; and 5, inotrope time. The results shown in FIGS. 8A-8C identify a surprising and unanticipated phenomenon where citrulline concentrations in the range of −200 micromolar appear to promote rather than prevent injury associated with cardiopulmonary bypass.

While not intending to be bound by a proposed mechanism, the inventors suggest that a negative feedback loop affects nitric oxide production, whereby high citrulline levels directly or indirectly inhibit nitric oxide synthase (eNOS) function. It is important to recall that citrulline is regenerated in the last step of NO production, making negative feedback by citrulline a reasonable hypothesis. Alternatively, an acute increase in NO production, which is known to have a negative feedback effect on NOS (Viziri and Wang, 1999) could explain the results. When studying a murine system, the inventors observed that increasing levels of citrulline initially facilitate nitric oxide production; however, continued increase in citrulline concentration results in diminished production of NO as measured via NO metabolites. The negative clinical effects in the >95% group for the Phase 3 trial may thus result from one or more negative feedback mechanisms. The negative feedback could result from either the effect of NO or the citrulline itself creating feedback on the system.

Analysis of global outcome data on the basis of dose administered was evaluated, with compelling—although somewhat counterintuitive—definition of a therapeutic window. In light of the results of the recent analyses, the preferred clinical target during cardiovascular bypass use is approximately 120 μmol, with an expectation of empirical serum levels well within the therapeutic window defined by the data. Irrespective of the endogenous level of L-citrulline in a given patient, it may be anticipated that the resulting range in clinical administration to pediatric CHD patients will be approximately 80-160 μmol. Maintaining plasma citrulline within an analogous therapeutic window may be expected to show benefits for non-infant pediatric or adult patients subjected to cardiopulmonary bypass, using analogous modes of administration. See U.S. Pat. Nos. 8,536,225, 10,525,026 and 10,265,286, incorporated herein by reference. Administration of citrulline so as to achieve plasma concentrations in the indicated range may also be expected to decrease the incidence of atrial fibrillation and other atrial arrhythmias that might otherwise accompany cardiopulmonary bypass in these patients.

Similarly, it may be anticipated that comparable therapeutic windows will apply to other conditions that respond to NO and/or citrulline therapy, including sickle cell disease, especially sickle cell crisis, subarachnoid hemorrhage, bronchopulmonary dysplasia, pulmonary hypertension, (including primary and secondary), hyperammonemia, asthma, and/or other urea cycle related conditions. Other representative disorders and conditions that may be expected to benefit from administration of citrulline within the therapeutic window include but are not limited to hepatitis (including hepatitis A, B and C), cirrhosis, sclerosis, asthma, especially acute asthma attack, Acute Respiratory Distress Syndrome (ARDS), bone marrow transplant toxicity in a subject undergoing bone marrow transplant, chemotherapy or other pharmaceutical therapy, sepsis, ethnic specific endothelial dysfunction, erectile dysfunction, priapism, necrotizing enterocolitis (NEC), increased oxidative stress, sepsis, hypoxia, hepatotoxin exposure, and combinations thereof. The therapeutic window targets plasma citruline concentration between μM, preferably greater than 70, 80, 90, or 100 μM but less than 160, 150, 140, or 130 μM. Preferably, citrulline administration is aimed at achieving a target plasma concentration of approximately 120 μM. Suitable modes of administration are described in, e.g., U.S. Pat. No. 8,536,225, incorporated herein by reference.

Evidence of Citrulline Efficacy in the Dose Window

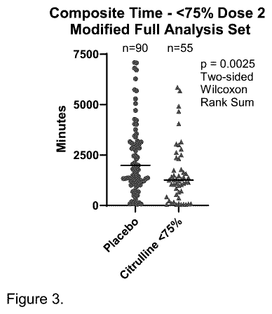

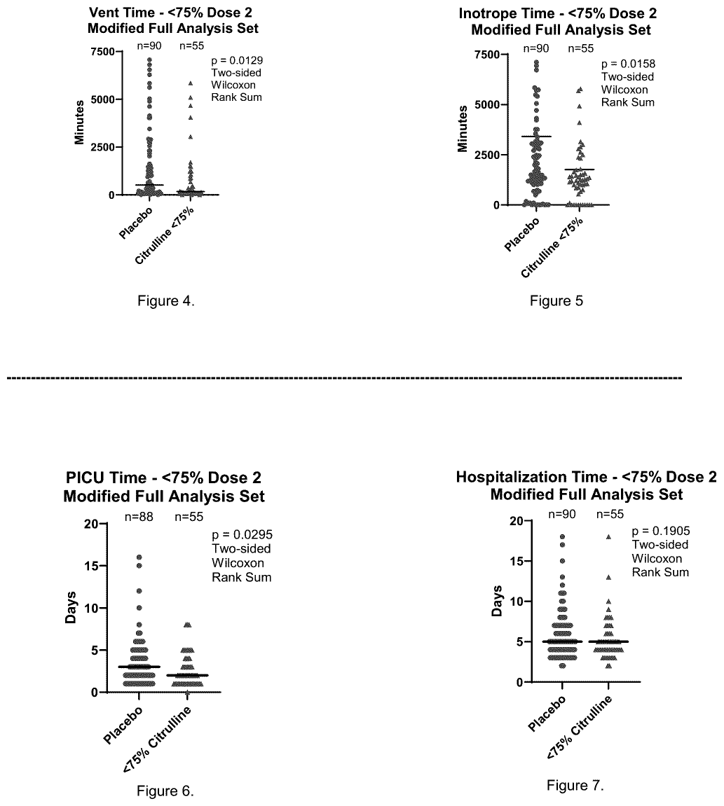

Further analysis of the data from the above-described Phase 3 clinical trial demonstrates efficacy across all of the ranges below 95% shown in FIG. 2. For illustrative purpose, patients receiving <75% of the target dose are shown in FIG. 3 for the composite variable, and below in FIGS. 4 and 5 for the duration of mechanical ventilation and the duration of inotrope therapy. Median values are shown in Table 2. The effect is robust, yielding substantially equivalent results across the entire range analyzed of <65% to <95% of the targeted dose.

As an example, FIG. 3 shows the results for the composite endpoint when the dose window upper limit is set at 75% of the calculated Dose 2. The composite endpoint shows highly significant differences between placebo and control in patients receiving <75% of the calculated intraoperative Dose 2 of citrulline. (Ten outlier data points above 7500 minutes are not shown, but are included in the analysis.) FIG. 3 shows that patients whose Dose 2 was <75% of the target dose (falling within the Dose 2 window established by the study) have a highly statistically significant reduction in the composite endpoint with p=0.0025, which meets the level of significance targeted (i.e., p<0.05). FIG. 4 shows the results for duration of mechanical ventilation for the modified full analysis set when the dose window upper limit is set at 75% of the calculated Dose 2. Five outlier data points above 7500 minutes are not shown, but are included in the analysis. FIG. 5 shows the results for duration of inotrope therapy for the modified full analysis set when the dose window upper limit is set at 75% of the calculated Dose 2. Eight outlier data points above 7500 minutes are not shown, but are included in the analysis. Table 2 provides an alternative presentation of the results for patients whose the dose window upper limit is set at 75% of the calculated Dose 2, by reporting the median time for each endpoint and the reduction in time observed in patients treated with the citrulline at or below 75% of the target level for Dose 2.

TABLE 2
Median values of end points for citrulline-
treated and placebo patients

| | Median | Median <75% | |
|---|---|---|---|
| | Placebo | Citrulline | |
| End Point | (minutes) | (minutes) | Reduction |
| Composite* | 2204.0 | 1267.0 | 42% |
| Mechanical | 574.5 | 170.0 | 70% |
| Ventilation | | | |
| Inotrope | 1717.0 | 1260.0 | 26% |

*Composite = the longer of the MV or InoTx

The primary endpoint for the trial was a composite comprising the duration of mechanical ventilation and duration of inotrope therapy in order to avoid the confounding of a direct measure of ICU stay by practical issues like bed availability. The benefit of citrulline treatment reflected in the composite variable, as shown in FIG. 3 and in Table 2, was mirrored in the measured clinical benefit of a shortened stay in the ICU (FIG. 6 and Table 3). There were fewer long-term hospitalizations in the citrulline-treated patients than in the placebo group; however the overall length of hospital stay was not significantly different between the two groups, as shown in FIG. 7. Both length of ICU stay and hospital stay were recorded in discrete units of one day, as reflected in the appearance of the graphs. FIG. 6 shows the duration of PICU/NICU stay for the modified full analysis set when the dose window upper limit is set at 75% of the calculated Dose 2. One outlier datapoint is excluded from the graph but not the analysis. Data was missing for two patients, who were excluded from the analysis. FIG. 7 shows the duration of hospital stay for the modified full analysis set when the dose window upper limit is set at 75% of the calculated Dose 2. One outlier datapoint is excluded from the graph but not from the analysis.

TABLE 3
Median duration of PICU/NICU stay

| | Median | Median <75% | |
|---|---|---|---|
| | Placebo | Citrulline | |
| End Point | (days) | (days) | Reduction |
| PICU/NICU | 3 | 2 | 33% |

1. A method of maintaining nitric oxide balance in a mammal in need thereof, comprising administering citrulline to the mammal such that the plasma citrulline concentration is maintained between 80-180 μM.

2. The method of claim 1, wherein citrulline is administered via intravenous bolus or infusion, orally, or via ultrafiltration in hemoconcentration replacement fluid.

3. The method of claim 1, wherein the mammal in need of nitric oxide balance suffers from hypocitrullinemia, is at risk for postoperative pulmonary hypertension, is at risk for acute right-sided heart failure, tricuspid regurgitation, systemic hypotension, myocardial ischemia, and/or increased airway resistance.

4. The method of claim 1, wherein the plasma citruline concentration is maintained between 80-160 μM.

5. The method of claim 1, wherein citrulline administration is aimed at achieving a target plasma concentration of approximately 120 μM.

6. A method for maintaining the coupling of endothelial nitric oxide synthase (eNOS) to reduce the incidence or severity of cardiopulmonary bypass-induced pulmonary injury due to free radical formation in a patient during cardiopulmonary bypass comprising administering an effective amount of citrulline to the patient, such that the plasma citrulline concentration is maintained between 80-180 μM.

7. The method of claim 6, comprising a step of administering citrulline to the patient during the surgery.

8. The method claim 6, wherein the citrulline administered during the surgery is added to the filtration.

9. The method of claim 6, wherein the citrulline administered during the surgery is added to hemoconcentration replacement fluid.

10. The method of claim 6, wherein the citrulline is added at about 80-180 μmol/L.

11. The method of claim 10, wherein the citrulline is added at about 80-160 μmol/L.

12. The method of claim 11, wherein the citrulline is added at about 120 μmol/L.

13. A method for maintaining the coupling of endothelial nitric oxide synthase (eNOS) to reduce the incidence or severity of sickle cell disease, especially sickle cell crisis, subarachnoid hemorrhage, bronchopulmonary dysplasia, pulmonary hypertension, (including primary and secondary), hyperammonemia, asthma, other urea cycle related conditions, hepatitis (including hepatitis A, B and C), cirrhosis, sclerosis, asthma, especially acute asthma attack, Acute Respiratory Distress Syndrome (ARDS), bone marrow transplant toxicity in a subject undergoing bone marrow transplant, chemotherapy or other pharmaceutical therapy, sepsis, ethnic specific endothelial dysfunction, erectile dysfunction, necrotizing enterocolitis (NEC), increased oxidative stress, sepsis, hypoxia, hepatotoxin exposure, and/or combinations thereof, said method comprising administering an effective amount of citrulline to the patient, such that the plasma citrulline concentration is maintained between 80-180 μM.

14. The method of claim 13, wherein citrulline is administered via intravenous bolus or infusion, or orally.

15. The method of claim 13, wherein the plasma citrulline concentration is maintained between 80-160 μM.

16. The method of claim 13, wherein citrulline administration is aimed at achieving a target plasma concentration of approximately 120 μM.
